(19) United States
(12) Patent Application Publication
(10) Pub. No.: US 2022/0071909 A1
(43) Pub. Date: Mar. 10, 2022

(54) MODIFIED RELEASE FORMULATIONS OF LEVODOPA

(71) Applicant: RUBICON RESEARCH PRIVATE LIMITED, Thane West (IN)

(72) Inventors: Pratibha S. Pilgaonkar, Thane West (IN); Ashwini H. Gadkari, Thane West (IN)

(21) Appl. No.: 17/445,992

(22) Filed: Aug. 26, 2021

(30) Foreign Application Priority Data
Aug. 26, 2020 (IN) 202021036841

(51) Int. Cl. A61K 9/20; A61K 31/197; A61K 9/00; A61P 25/16

(52) U.S. Cl. CPC A61K 9/2086; A61K 31/197; A61P 25/16; A61K 9/2054; A61K 9/0053; A61K 9/2031

(57) ABSTRACT

The present invention relates to modified release formulations of levodopa. Particularly the invention relates to modified release formulation of levodopa that offers reduced dosing frequency.

### FIELD OF THE INVENTION

The present invention relates to modified release formulations of levodopa. Particularly the invention relates to modified release formulation of levodopa that offers reduced dosing frequency.

### BACKGROUND OF THE INVENTION

Parkinson's disease is a long-term, degenerative, neurological disease that causes a person to lose control over some body functions. It affects the nerve cells in the brain that produce dopamine. Parkinson's disease symptoms include muscle rigidity, tremors, and changes in speech and gait. The symptoms usually begin gradually and get worse over time. As the disease progresses, people may have difficulty walking and talking. They may also have mental and behavioural changes, sleep problems, depression, memory difficulties, and fatigue.

Levodopa (LD) the precursor to dopamine has been the mainstay for the treatment of Parkinson's disease. It not only controls the symptoms but may slow the disease progression as well. Levodopa crosses the blood brain barrier and is rapidly converted to dopamine, thereby alleviating the symptoms of Parkinson's disease caused by reduced levels of dopamine. One of the major issues associated with levodopa treatment is that it requires frequent dosing—often about three to eight times during waking hours—to maintain an efficacious drug concentration in the plasma because of a short half-life (˜1 hour), which is caused by extensive metabolism via peripheral decarboxylase. Thus, when levodopa is administered alone, large doses are required because only a small portion is transported to the brain unchanged.

Levodopa is therefore commonly co-administered with carbidopa (CD), an aromatic amino acid decarboxylase inhibitor, to reduce the extensive peripheral degradation. Decarboxylase inhibitor such as carbidopa primarily block levodopa metabolism in the periphery, thereby reducing the rate of the first-pass metabolism and slowing the plasma clearance of levodopa. An additional benefit of this reduced peripheral decarboxylation of levodopa to dopamine is the amelioration of the characteristic peripheral side effects of dopamine (i.e., nausea, vomiting, and anorexia).

Yet however a frequent dosing schedule is still required. Aside from inconvenience and issues regarding the lack of compliance, the frequent dosing schedule results in more peaks and troughs in the plasma concentration time profile and pulsatile stimulation of dopamine receptors, which is believed to result in fluctuating motor performance and induction of dyskinesia.

Patients with relatively early Parkinson's disease, when placed on carbidopa-levodopa immediate release three or four times daily, typically experience a robust response, with good improvement in bradykinesia and rigidity, and this response persists through the day despite levodopa's short half-life. Presumably, this is because levodopa is taken up by remaining dopamine neurons, decarboxylated to dopamine, stored intraneuronally and then slowly released into the synapse over time to provide antiparkinsonian benefit. However, as more and more dopamine neurons degenerate, this storage and release capacity or buffering capacity is progressively lost, and patients experience a clinical response that more and more closely mirrors the peripheral pharmacokinetics of levodopa. After a few years of Carbidopa-Levodopa therapy, many patients find that the clinical benefit of a dose lasts a few hours and then wears off with a return of motor symptoms.

Chronic levodopa therapy is thus often associated with the development of motor function complications such as ‘on-off phenomena’, ‘wearing-off’, and dyskinetic movements. The advanced form of motor fluctuations (‘on-off’ phenomenon) is characterized by unpredictable swings from mobility to immobility. Although several factors contribute to the development of motor complications in levodopa therapy, pulsatile stimulation of dopamine receptors and high levodopa doses are considered key factors. Continuous stimulation of striatal dopaminergic receptors thus remains the goal and even currently is an unmet need of oral pharmacotherapy for Parkinson's disease. Therefore, treatment strategies that reduce fluctuation in plasma concentrations and thereby stabilize motor performance are desired.

The problem of peaks and troughs of levodopa in the plasma with immediate release formulation and corresponding peaks and troughs of dopamine in the synaptic cleft between presynaptic and postsynaptic dopaminergic neurons especially with the progression of Parkinson Disease due to diminished buffering capacity of the dopaminergic neurons is typically seen with levodopa therapy. As a result, all orally levodopa treated Parkinson's disease patients sooner or later, but inevitably experience quality of life affecting fluctuations of movements. These so-called ‘motor complications’ are characterized by intervals with good movement behaviour, the so-called ‘ON’-time. The term ‘OFF’-states describe periods with the onset of motor symptoms when the Levodopa effect starts to vane. A transient overstimulation of postsynaptic dopamine receptors is believed to be one of the main reasons for the manifestation of involuntary movements, termed as ‘dyskinesias’ that are experienced. Many patients (between 40% and 100%) have been reported to develop fluctuating motor and non-motor symptoms (wearing-off and on-off phenomena) and dyskinesias with continued use of Levodopa. Motor fluctuations can be noted as soon as 5-6 months after treatment initiation, particularly with large doses (≥600 mg/d). After 2 years of carbidopa-levodopa therapy, as few as 20% of patients experience sustained benefits. More than 70% of patients experience on-off fluctuations and dyskinesias after 5 years. As wearing-off becomes more frequent, Levodopa doses need to be given in increasing frequency to achieve a sufficient clinical response. The fluctuation of pharmacodynamics response to Levodopa between wearing off and levodopa-induced dyskinesias leads to a narrower optimal therapeutic window with increasing probability of overdosage and underdosage.

It is generally believed that a more continuous peripheral availability of levodopa resulting in turn in continuous availability of dopamine at the dopaminergic neurons can result in a steadier and longer clinical improvement without the development of dyskinesia.

Over the years, strategies have been tried to increase the duration of bioavailability in Parkinson's disease patients. Reducing the interval between Levodopa doses through the administration of controlled release formulations was one of the approaches utilized to solve the ‘wearing off’ problem encountered with Levodopa therapy. Thus, controlled release formulations were developed with the intention of delivering Levodopa to the brain in such a manner that little or no fluctuations in Levodopa concentrations would occur. Unfortunately, the intention was not fulfilled, as patients still experienced motor complications because of plasma fluctuations.

Either their longer duration to onset of effect compared with immediate release (IR) levodopa, or less consistent clinical responses, or their dosing lacking the fidelity to meet the nuanced needs of more advanced fluctuating patients were the issues.

Administration of Sinemet® CR for example needs to be frequent usually at intervals ranging from 4 to 8 hours during the waking day, primarily because Sinemet® CR is less systemically bioavailable than Sinemet® and therefore may require increased daily doses to achieve the same level of symptomatic relief as provided by Sinemet®. Rytary® on the other hand being only 70% bioavailable (based on concentration area under the curve [AUC]) compared to immediate release carbidopa/levodopa (Sinemet® IR), needs an initial dosing frequency of three times daily, but a maximum dosing frequency of five times daily, if tolerated, is suggested to maximize symptomatic control.

Both Sinemet® CR and Rytary® are thus conventional matrix type and multiparticulate type drug delivery systems respectively that are less bioavailable than the immediate release levodopa formulation. This results in the need to administer increased daily doses of the active to achieve the same relief as provided by the immediate release formulation resulting in reduced patient compliance and even exacerbation of motor complications and dyskinesia. A frequent dosing schedule results in larger fluctuations between peaks and troughs in levodopa plasma concentration-time profile which may cause variable motor performance and induction of dyskinesia. The desired continuous controlled oral delivery of levodopa has been a challenge with these conventional controlled release dosage forms due to the variable in vivo absorption of levodopa from these formulations primarily because the absorption of levodopa is limited to the narrow absorption window near the proximal small intestine where the transporters for levodopa are located.

Conventional controlled or extended release matrix or multiparticulate systems may therefore have limited use in the case of levodopa therapy as only the drug substance released in the region preceding and in close vicinity of the absorption window would be available for absorption. After crossing the absorption window, the released drug substance will show very little or no absorption. This phenomenon drastically limits the success of the commercially available conventional controlled delivery systems.

A continuous administration of levodopa to the upper gastrointestinal tract near the apparent narrow absorption window thereof is therefore essential to facilitate a prolonged and continuous absorption phase of the drug near the absorption window and maintain sustained therapeutic levels. The present inventors have developed after tremendous efforts modified release formulation of levodopa which is gastroretentive in nature. Such a gastroretentive system can avoid the significant heterogeneity throughout the gastrointestinal tract including pH, microbial flora, gastrointestinal transit time, enzymatic activity, aqueous environment, and surface area, all of which may influence absorption. Such a formulation of levodopa that is retained in the stomach for longer periods of time with controlled drug release profile achieves the objective of reduced frequency of dosing.

Where conventional controlled release dosage forms thus fail to provide a constant supply of levodopa at its absorption site, gastroretentive dosage forms could provide lesser fluctuating plasma concentrations and more complete absorption. Better spatial and temporal targeting with gastroretentive type of modified release formulations can decrease the frequency of the administration of levodopa doses. Such a gastroretentive formulation may also have acceptable side effect characteristics with well controlled release and absorption of the active resulting in an improved pharmacokinetic profile with reduced peaks and troughs of blood levels.

### DETAILED DESCRIPTION OF THE INVENTION

The present invention provides modified release formulations of levodopa. The modified release formulations of levodopa of the present invention are in the form of a gastroretentive dosage form. The compositions of the present invention swell voluminously to cause the size of the dosage form to increase such that the swollen dosage form does not pass through the pyloric sphincter resulting in retention of the dosage form for a prolonged period of time. The modified release gastroretentive formulation of the present invention provides a continuous supply of levodopa such that the dosing frequency otherwise required for the levodopa-carbidopa therapy is reduced. The formulations of the present invention thereby need to be administered only once or twice a day to achieve an acceptable therapeutic outcome.

The modified release formulation of the present invention comprises the active agent, levodopa. The formulation further comprises at least one decarboxylase inhibitor. In one embodiment, the decarboxylase inhibitor employed is carbidopa. In one embodiment, the active agent can be present in the form of any hydrate. In a further embodiment, the decarboxylase inhibitor can be present in the form of any hydrate. In one embodiment, levodopa is present in the composition of the present invention in an amount of about 1% to about 60% by weight of the dosage form. In one embodiment, carbidopa is present in the composition of the present invention in an amount of about 1% to about 60% by weight of the dosage form. In one embodiment, levodopa may be present in the dosage form of the present invention in an amount of from about 50 mg to about 1000 mg. In a further embodiment, levodopa may be present in the dosage form of the present invention in an amount of 200 mg. In another embodiment, carbidopa may be present in the dosage form of the present invention in an amount of from about 2 mg to about 500 mg. In a further embodiment, carbidopa may be present in the dosage form of the present invention in an amount of 200 mg.

In a further embodiment, the modified release formulation of the present invention further comprises a swelling agent. In one embodiment, the swelling agent employed in the present invention includes, but is not limited to, polyethylene oxide and the like. In another embodiment, the swelling agent employed in the present invention includes, but is not limited to, polyethylene oxide, hydroxypropyl methyl cellulose, higher viscosity (Methocel K100M) and the like or any combination thereof. In a further embodiment, the swelling agent employed in the present invention includes, but is not limited to, polyethylene oxide, hydroxypropyl methyl cellulose, higher viscosity (Methocel K100M), hydroxy ethyl cellulose and the like or any combination thereof. In one embodiment the composition of the present invention comprises about 10% to about 80% by weight of the composition. In a further embodiment, the composition of the present invention comprises about 20% to about 70% by weight of the composition.

The formulations of the present invention may further comprise one or more release retardants. In one embodiment, the release retardants that may be employed include, but are not limited to, hydroxypropyl methyl cellulose, lower viscosity (Methocel K4M) and the like. In a further embodiment the release retardant is present in the composition of the present invention at about 1% to about 50% by weight of the composition. In another embodiment, the release retardant is present in the composition of the present invention at about 1% to about 40% by weight of the composition.

The formulation may further comprise at least one pharmaceutically acceptable excipient, such as, but not limited to, diluents, binders, glidants, lubricants, disintegrants, effervescent couple, antioxidants, preservatives and the like or any combinations thereof. Suitable diluents that may be employed include, but are not limited to, microcrystalline cellulose, lactose, dicalcium phosphate, and the like or any combinations thereof. Suitable binders that may be employed include, but are not limited to, polyvinyl pyrrolidone, copovidone, hydroxypropyl cellulose and the like or any combinations thereof. Suitable lubricants that may be employed include, but are not limited to, magnesium stearate, calcium stearate, stearic acid, talc, and sodium stearyl fumarate and the like or any combinations thereof. Suitable glidants that may be employed include, but are not limited to, colloidal silicon dioxide, talc, or any combinations thereof. Suitable disintegrants that may be employed include, but are not limited to, crospovidone and the like. Suitable effervescent couple such as sodium bicarbonate and citric acid and the like may be employed. Suitable preservative such as, but not limited to, methyl paraben or propyl paraben and the like or combination thereof may be employed. In one embodiment, one or more pharmaceutically acceptable excipients are present in an amount of more than about 10% of the total weight of the composition. In another embodiment, one or more pharmaceutically acceptable excipients are present in an amount of about 10% to about 90% by weight of the composition.

In another embodiment, the formulation of the present invention is in the form of a bi-layered system. In another embodiment, the dosage form of the present invention may be in the form of a tablet. In a further embodiment, the modified release formulation of the present invention is in the form of a bi-layered tablet dosage form. In another embodiment, the modified release formulation of the present invention is in the form of a bi-layered tablet dosage form comprising active layer and gastroretentive layer or placebo layer. In an embodiment, the formulation of the present invention is not a multiparticulate system. In a further embodiment, the layers of the layered dosage form are in the form of a matrix. In another embodiment, the dosage form of the present invention may be coated. In a further embodiment, the dosage form of the present invention may be uncoated.

The term “composition” or “formulation” or “dosage form” or “preparation” has been employed interchangeably for the purpose of the present invention and mean that it is a pharmaceutical formulation which is suitable for administration to a patient. For the purpose of the present invention, the terms “controlled release” or “sustained release” or “extended release” or “modified release” or “prolonged release” have been used interchangeably and mean broadly that the active agent is released at a predetermined rate that is different or slower than immediate release.

In one embodiment, the formulation of the present invention is a gastro-retentive system. In a further embodiment, the modified release formulation of the present invention releases levodopa over a period of 4 hours or more. In another embodiment, the modified release formulation of the present invention releases levodopa over a period of 6 hours or more.

The formulations of the present invention are prepared by process such as direct compression, wet granulation, dry granulation and the like or any combinations thereof. The present invention provides process for preparation of the gastro-retentive formulations. In an embodiment the dosage form of the present invention is retained in the upper gastrointestinal tract for more than about 2 hours. In another embodiment the dosage form of the present invention is retained in the upper gastrointestinal tract for about 2 hours to 8 hours. In a further embodiment, the dosage form of the present invention is retained in the upper gastrointestinal tract for more than about 4 hours. In yet another embodiment, the dosage form of the present invention is retained in the upper gastrointestinal tract for about 4 hours to 8 hours. In one embodiment, the bi-layered tablet dosage form of the present invention has the gastroretentive layer as not less than 60% of the total tablet weight. In another embodiment, the swelling index of the dosage forms of the present invention were evaluated. In one embodiment, the swelling index of the dosage form of the present invention was greater than 60% at one hour. In a further embodiment, the swelling index of the dosage form of the present invention is greater than 70% at one hour. In one embodiment, the swelling index of the dosage form of the present invention is greater than 200% at eight hours.

In another embodiment, the formulation of the present invention requires only once a day or twice a day administration. In a further embodiment, the formulation of the present invention presents a reduced fluctuation index. In another embodiment, the formulation of the present invention presents a reduced peak trough ratio.

In a further embodiment, the modified release formulation of the present invention is employed for the treatment of Parkinson's disease. In another embodiment, the modified release formulation of the present invention is employed for the treatment of any disease or disorder relating to reduced dopamine levels.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed, but that the invention will include all embodiments falling within the scope thereof. Details of the present invention, including its objects and advantages, are provided in the non-limiting exemplary illustrations below.

### EXAMPLES

### Example 1: Carbidopa-Levodopa Modified Release Gastroretentive Tablet

| | |
|---|---|
| Ingredient | mg/tab |

Active layer

| | |
|---|---|
| Carbidopa | 50 |
| Levodopa | 200 |
| Hydroxypropyl methyl cellulose, low | 30 |
| viscosity (Methocel K4M) | |
| Hydroxypropyl cellulose | 25 |
| Microcrystalline cellulose | 10.5 |
| Methyl paraben | 0.2 |
| Propyl paraben | 0.05 |
| FD&C Red 40Al. Lake | 0.25 |
| Magnesium stearate | 4 |
| Isopropyl alcohol | q.s |
| Total | 320 |

Gastroretentive layer

| | |
|---|---|
| Polyethylene oxide | 120 |
| Hydroxypropyl methyl cellulose, | 120 |
| higher viscosity (Methocel K100M) | |
| Hydroxy ethyl cellulose | 60 |
| Crospovidone | 120 |
| Microcrystalline cellulose | 40 |
| Polyvinyl pyrrolidone | 30 |
| Copovidone | 10.4 |
| Methyl paraben | 0.55 |
| Propyl paraben | 0.05 |
| Sodium bicarbonate | 30 |
| Citric acid | 9 |
| Isopropyl alcohol | qs |
| Water | qs |
| Mg. stearate | 5 |
| Total | 545 |
| Total tablet weight | 865 |

Procedure: The components of the active layer except magnesium stearate are blended and granulated with hydroxy propyl cellulose solution in isopropyl alcohol. The granules are then lubricated with magnesium stearate to form the active layer blend. The components of the gastroretentive layer except effervescent couple, magnesium stearate and copovidone are blended and granulated with povidone. The remaining excipients are then blended with these granules to form the gastroretentive layer blend. The active layer blend and the gastroretentive layer blend were then compressed into bi-layered tablets.

### Example 2: Carbidopa-Levodopa Modified Release Gastro-Retentive Tablet

| | |
|---|---|
| Ingredient | mg/tab |

Active layer

| | |
|---|---|
| Carbidopa | 50 |
| Levodopa | 200 |
| Hydroxypropyl methyl cellulose, low | 30 |
| viscosity (Methocel K4M) | |
| Hydroxypropyl cellulose | 25 |
| Microcrystalline cellulose | 10.5 |
| Methyl paraben | 0.2 |
| Propyl paraben | 0.05 |
| FD&C Red 40Al. Lake | 0.25 |
| Magnesium stearate | 4 |
| Isopropyl alcohol | q.s |
| Total | 320 |

Gastro-retentive layer

| | |
|---|---|
| Polyethylene oxide | 150 |
| Ethyl Cellulose | 60 |
| Hydroxypropyl methyl cellulose, | 150 |
| higher viscosity (Methocel K100M) | |
| Hydroxy ethyl cellulose | 70 |
| Crospovidone | 150 |
| Microcrystalline cellulose | 40 |
| Polyvinyl pyrrolidone | 40 |
| Copovidone | 50 |
| Methyl paraben | 0.9 |
| Propyl paraben | 0.1 |
| Sodium bicarbonate | 40 |
| Citric acid | 10 |
| Isopropyl alcohol | qs |
| Water | qs |
| Mg. stearate | 5 |
| Total | 545 |
| Total tablet weight | 865 |

Procedure: The components of the active layer except magnesium stearate are blended and granulated with hydroxy propyl cellulose solution in isopropyl alcohol. The granules are then lubricated with magnesium stearate to form the active layer blend. The components of the gastroretentive layer except effervescent couple, magnesium stearate and copovidone are blended and granulated with povidone. The remaining excipients are then blended with these granules to form the gastroretentive layer blend. The active layer blend and the gastroretentive layer blend were then compressed into bi-layered tablets.

### Example 3: Dissolution Data for Bilayer Tablet Comprising Carbidopa-Levodopa of Example 2

The dissolution evaluation was conducted using the following—

Media: 0.001 N HCl (pH 3.0)

Media Volume: 900 ml

Type of Dissolution Apparatus: Type I

RPM: 100

| | |
|---|---|
| Time (Hrs) | % Drug Release |

Carbidopa 50 mg

| | |
|---|---|
| 0.5 | 5-10% |
| 2 | 10-20% |
| 4 | 20-30% |
| 8 | 40-50% |
| 12 | 50-60% |
| 24 | 70-80% |

Levodopa 200 mg

| | |
|---|---|
| 0.5 | 5-10% |
| 2 | 10-20% |
| 4 | 20-30% |
| 8 | 40-50% |
| 12 | 50-60% |
| 24 | 70-80% |

### Example 4: Swelling Index of the Bilayer Tablet Comprising Carbidopa-Levodopa of Example 2

Swelling index of tablets were evaluated. Study was performed in USP Dissolution apparatus II, with paddle speed at 25 RPM. Media used for study was 500 mL of pH 3 (0.001 N HCl) for 1 hrs, 2 hrs, 4 hrs, 6 hrs maintaining temperature at 37° C.±0.5° C. Initial weights and physical parameters such as tablet diameter and thickness were noted. Swelling index was calculated based on volume by tablets as follows:

$$Swelling Index = \frac{Final {Volume}_{( at T )} - Initial {volume}_{( at T 0 )}}{Initial Volume} \times 100$$

| | Media |
|---|---|
| | 0.001N HCl (pH 3), Volume- 500 ml, |
| | Paddle (Type 2 Apparatus), 25 RPM |
| Time | % Swelling index |
| 1 hr | 80-90% |
| 2 hr | 110-120% |
| 4 hr | 155-165% |
| 6 hr | 180-190% |
| 8 hr | More than 200% |

We claim:

1) A gastroretentive dosage form of levodopa comprising:
(a) levodopa
(b) carbidopa
(c) swelling agent and
(d) release retardant.

2) The gastroretentive dosage form of claim 1 wherein the dosage form is a multi-layered dosage form.

3) The gastroretentive dosage form of claim 2 wherein the multi-layered dosage form comprises:
(a) a drug layer comprising levodopa, carbidopa and a release retardant,
(b) a swellable gastro-retentive layer comprising swelling agent.

4) The gastroretentive dosage form of claim 3, wherein the dosage form exhibits a swelling index of at least 60% in one hour.

5) The gastro-retentive dosage form of claim 2, wherein the dosage form is a bi-layered tablet.

6) The gastro-retentive dosage form of claim 1, wherein the swelling agent is polyethylene oxide.

7) The gastro-retentive dosage form of claim 6, wherein the swelling agent is polyethylene oxide and hydroxy propyl methyl cellulose, higher viscosity.

8) The gastro-retentive dosage form of claim 1, wherein the swelling agent is hydroxy propyl methyl cellulose, lower viscosity.

9) The gastro-retentive dosage form of claim 1, wherein the dosage form further comprises one or more pharmaceutically acceptable excipients comprising diluents, binders, disintegrants, glidants, lubricants, coloring agents, gas generating agents or coating agents.
